(19) United States
(12) Patent Application Publication
(10) Pub. No.: US 2026/0199616 A1
(43) Pub. Date: Jul. 16, 2026

(54) SELF-RESCUE ANTI-ASPHYXIATION DEVICE

(71) Applicant: Jinhua Choking device Medical Supplies Co., Ltd., Jinhua City (CN)

(72) Inventor: Meng Zheng, Yuyao City (CN)

(73) Assignee: Jinhua Choking device Medical Supplies Co., Ltd., Jinhua City (CN)

(21) Appl. No.: 19/053,967

(22) Filed: Feb. 14, 2025

(30) Foreign Application Priority Data
Jan. 14, 2025 (CN) 202520078517.6

(51) Int. Cl. A61M 16/00; A61M 16/06

(52) U.S. Cl. CPC A61M 16/0057; A61M 16/0622; A61M 2205/0216; A61M 2205/125; A61M 2210/1032

(57) ABSTRACT

The present invention discloses a self-rescue anti-asphyxiation device, including a suction main body and a mask, where the suction main body includes a cylinder body and a cover body which are hermetically connected, the cylinder body includes a cylinder shell having an elastic folding capability, a suction chamber is formed between the cylinder body and the cover body, the cover body is provided with a suction pipe and an exhaust hole which are communicated with the suction chamber, the cover body is provided with a one-way exhaust valve corresponding to the exhaust hole, the exhaust hole and the one-way exhaust valve are arranged on an end face of one side of the cover body far away from the cylinder body, and the suction pipe is integrally arranged on a side wall of the cover body; and the mask is provided with a connecting sleeve, the connecting sleeve is fixedly connected with the suction pipe in an inserting manner, the mask is provided with a suction hole communicated with an inner cavity of the connecting sleeve in a penetrating manner, and the mask is provided with a one-way suction valve corresponding to the suction hole. The device has the effects that a user can conveniently observe the real-time working condition of the one-way exhaust valve, the one-way exhaust valve can be conveniently and quickly replaced or cleaned, and the space occupied when the device is stored is reduced.

### REFERENCE TO PRIOR APPLICATION

This application claims priority to Chinese Patent Application 202520078517.6, filed on Jan. 14, 2025, which is incorporated herein by reference.

### TECHNICAL FIELD

The present invention relates to the technical field of medical appliances, in particular to a self-rescue anti-asphyxiation device.

### BACKGROUND

The existing Chinese patent with the publication number of CN221243594U discloses a self-rescuable asphyxiation first-aid device, which includes a cylinder shell, a choke valve, and a mask, where one end of the cylinder shell is provided with a notch, a piston assembly is arranged inside the cylinder shell, the other end of the cylinder shell is sleeved with a rear cover, and the rear cover is used for guiding when the piston assembly slides; the choke valve is arranged on the piston assembly, and is used for switching on or off airflow; and the mask is arranged on a lower side of the notch.

However, the above asphyxiation first-aid device has the following disadvantages that the choke valve of the asphyxiation first-aid device is arranged on the piston assembly, when the choke valve is damaged, the choke valve is inconvenient to replace, moreover, a user cannot observe the installation completeness of the choke valve at the first time during emergency use, and in addition, the cylinder shell of the asphyxiation first-aid device is generally made of a hard material, so that the space occupied during storage is large, and the asphyxiation first-aid device is inconvenient to store and carry. Therefore, the asphyxiation first-aid device needs to be improved urgently.

### SUMMARY

The present invention aims to provide a self-rescue anti-asphyxiation device, which has the effects that a user can conveniently observe the real-time working condition of a one-way exhaust valve, the one-way exhaust valve can be conveniently and quickly replaced or cleaned, and the space occupied when the device is stored is reduced.

The technical purpose of the present invention is achieved through the following technical solution. The self-rescue anti-asphyxiation device includes a suction main body and a mask, where the suction main body includes a cylinder body and a cover body which are hermetically connected, the cylinder body includes a cylinder shell having an elastic folding capability, a suction chamber is formed between the cylinder body and the cover body, the cover body is provided with a suction pipe and an exhaust hole which are communicated with the suction chamber, the cover body is provided with a one-way exhaust valve corresponding to the exhaust hole, the exhaust hole and the one-way exhaust valve are arranged on an end face of one side of the cover body far away from the cylinder body, and the suction pipe is integrally arranged on a side wall of the cover body; and

the mask is provided with a connecting sleeve, the connecting sleeve is fixedly connected with the suction pipe in an inserting manner, the mask is provided with a suction hole communicated with an inner cavity of the connecting sleeve in a penetrating manner, and the mask is provided with a one-way suction valve corresponding to the suction hole.

By adopting the above technical solution, in the present invention, the exhaust hole and the one-way exhaust valve are arranged on the end face of one side of the cover body far away from the cylinder body, and the suction pipe is integrally arranged on the side wall of the cover body, so that this layout is favorable for a user to observe the use condition of the one-way exhaust valve in real time when the anti-asphyxiation device is used for self-rescue, and interference impurities near the exhaust hole can be conveniently cleaned by the user in time. Meanwhile, when the one-way exhaust valve is damaged, the exposed one-way exhaust valve is easier to replace. In addition, when the device is stored, the cylinder shell can be compressed to be folded for storage, so that the space occupied when the device is stored is greatly reduced. The device has the effects that a user can conveniently observe the real-time working condition of the one-way exhaust valve, the one-way exhaust valve can be conveniently and quickly replaced or cleaned, and the space occupied when the device is stored is reduced.

A further arrangement of the present invention is that the cylinder body includes a hard threaded connecting part integrally connected with the cylinder shell, the cover body is integrally provided with a threaded matching part corresponding to the hard threaded connecting part, and the hard threaded connecting part is in threaded connection with the threaded matching part.

By adopting the above technical solution, the cylinder body and the cover body can be quickly disassemble or assembled.

A further arrangement of the present invention is that both the one-way exhaust valve and the one-way suction valve include a diaphragm and a mandrel integrally connected to a middle of the diaphragm, the mask and the cover body are provided with installation holes corresponding to the mandrels, and the mandrels are movably arranged in the corresponding installation holes.

By adopting the above technical solution, the mandrels are movably arranged in the corresponding installation holes, and the efficient sealing of the one-way exhaust valve or the one-way suction valve can be realized by using the elastic deformation capability and better sealing adhesion of the diaphragm.

A further arrangement of the present invention is that an annular flange is obliquely arranged on a periphery of the diaphragm in an extending manner toward the cover body or the mask.

By adopting the above technical solution, the addition of the annular flange can prevent the situation that the diaphragm is difficult to separate because the diaphragm is attached to the surface of the mask or the cover body in a large area.

A further arrangement of the present invention is that the cover body is provided with an annular protective sleeve around the exhaust hole, the one-way exhaust valve is arranged in the annular protective sleeve and is in shielding fit with the corresponding exhaust hole, and an airflow gap is reserved between a side wall of the one-way exhaust valve and the annular protective sleeve.

By adopting the above technical solution, the annular protective sleeve can protect the one-way exhaust valve, and meanwhile, the reservation of the airflow gap can also ensure that the airflow in the cylinder body can be smoothly exhausted from the exhaust hole.

A further arrangement of the present invention is that the cylinder shell is designed as a rubber bellows.

By adopting the above technical solution, the rubber bellows is provided with a plurality of skirt edges, so that the pressing and folding capability of the cylinder shell can be improved.

A further arrangement of the present invention is that a handle part is integrally connected to one end of the cylinder shell far away from the cover body.

By adopting the above technical solution, the addition of the handle part can improve the pressing convenience of a user on the cylinder shell and facilitate the operation convenience of the user.

A further arrangement of the present invention is that a face fitting ring made of a flexible material is connected to an open end of the mask.

By adopting the above technical solution, the addition of the face fitting ring can effectively improve the wrapping property and the comfort of the fitting between the device and a human face.

A further arrangement of the present invention is that the mask is internally provided with a plurality of filter plates, and each of the filter plates is provided with a plurality of air passage holes.

By adopting the above technical solution, the addition of the filter plates can prevent insects or dust from entering the cylinder body, and meanwhile, if a user sneezes when wearing the mask, foreign matters can be effectively blocked on one side of the mask far away from the cylinder body.

A further arrangement of the present invention is that two filter plates are provided, the two filter plates are arranged in the mask at an interval, and the air passage holes on the two filter plates are arranged in a staggered manner.

By adopting the above technical solution, the blocking property of the filter plates to the foreign matters can be further improved.

In conclusion, the present invention has the following beneficial effects.

The exhaust hole and the one-way exhaust valve are arranged on the end face of one side of the cover body far away from the cylinder body, and the suction pipe is integrally arranged on the side wall of the cover body, so that this layout is favorable for a user to observe the use condition of the one-way exhaust valve in real time when the anti-asphyxiation device is used for self-rescue, and interference impurities near the exhaust hole can be conveniently cleaned by the user in time. Meanwhile, when the one-way exhaust valve is damaged, the exposed one-way exhaust valve is easier to replace. In addition, when the device is stored, the cylinder shell can be compressed to be folded for storage, so that the space occupied when the device is stored is greatly reduced. The device has the effects that a user can conveniently observe the real-time working condition of the one-way exhaust valve, the one-way exhaust valve can be conveniently and quickly replaced or cleaned, and the space occupied when the device is stored is reduced.

### BRIEF DESCRIPTION OF DRAWINGS

FIG. 1 is an overall structure diagram of an embodiment 1 of the present invention;

FIG. 2 is a longitudinal sectional view of an embodiment 1 of the present invention;

FIG. 3 is a partial enlarged view of an area A in FIG. 2 of the present invention;

FIG. 4 is a longitudinal sectional view of two types of masks of an embodiment 2 of the present invention; and

FIG. 5 is a partial enlarged view of an area B in FIG. 4 of the present invention.

Reference numerals: 1: suction main body; 11: suction chamber; 2: cylinder body; 21: cylinder shell; 22: hard threaded connecting part; 23: handle part; 3: cover body; 31: suction pipe; 32: exhaust hole; 33: one-way exhaust valve; 331: diaphragm; 332: mandrel; 333: annular flange; 34: threaded matching part; 35: annular protective sleeve; 351: airflow gap; 4: mask; 41: connecting sleeve; 42: suction hole; 43: one-way suction valve; 44: installation hole; 45: face fitting ring; 46: filter plate; and 461: air passage hole.

### DETAILED DESCRIPTION OF EMBODIMENTS

The present invention is further described below with reference to the accompanying drawings.

### Embodiment 1

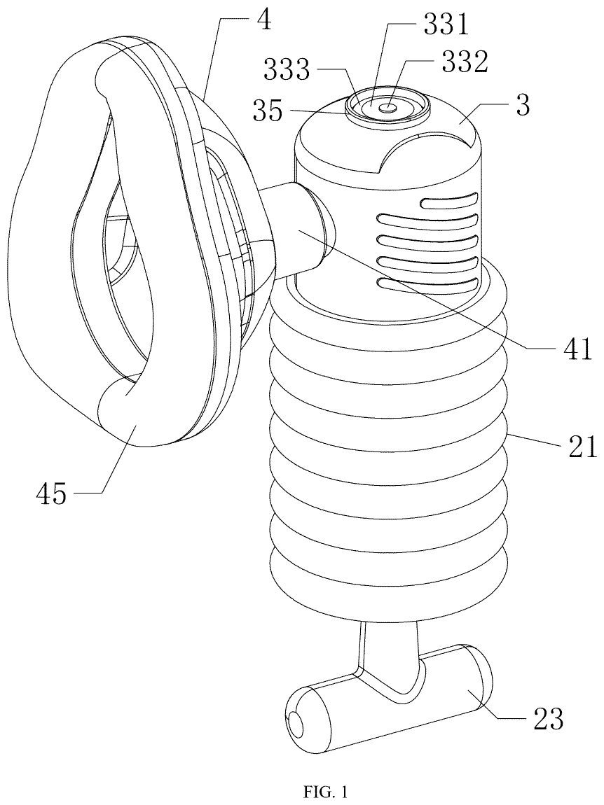

As shown in FIGS. 1-3, a self-rescue anti-asphyxiation device includes a suction main body 1 and a mask 4, where the suction main body 1 includes a cylinder body 2 and a cover body 3 which are hermetically connected, the cylinder body 2 includes a cylinder shell 21 having an elastic folding capability, a suction chamber 11 is formed between the cylinder body 2 and the cover body 3, the cover body 3 is provided with a suction pipe 31 and an exhaust hole 32 which are communicated with the suction chamber 11, the cover body 3 is provided with a one-way exhaust valve 33 corresponding to the exhaust hole 32, the exhaust hole 32 and the one-way exhaust valve 33 are arranged on an end face of one side of the cover body 3 far away from the cylinder body 2, and the suction pipe 31 is integrally arranged on a side wall of the cover body 3; and the mask 4 is provided with a connecting sleeve 41, the connecting sleeve 41 is fixedly connected with the suction pipe 31 in an inserting manner, the mask 4 is provided with a suction hole 42 communicated with an inner cavity of the connecting sleeve 41 in a penetrating manner, and the mask 4 is provided with a one-way suction valve 43 corresponding to the suction hole 42; the cylinder shell 21 is designed as a rubber bellows, the rubber bellows is provided with a plurality of skirt edges, so that the pressing and folding capability of the cylinder shell 21 can be improved; a handle part 23 is integrally connected to one end of the cylinder shell 21 far away from the cover body 3, and the addition of the handle part 23 can improve the pressing convenience of a user on the cylinder shell 21 and facilitate the operation convenience of the user; and a face fitting ring 45 made of a flexible material is connected to an open end of the mask 4, and the addition of the face fitting ring 45 can effectively improve the wrapping property and the comfort of the fitting between the device and a human face.

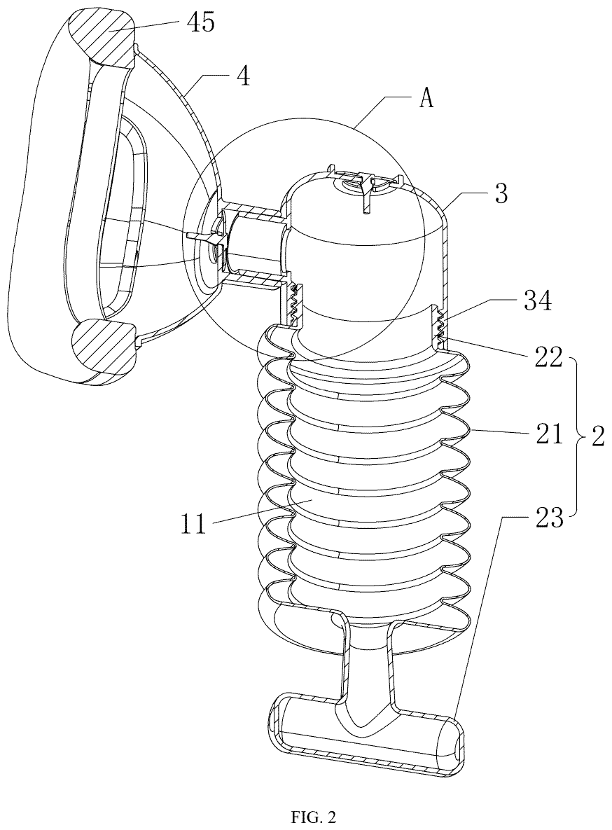

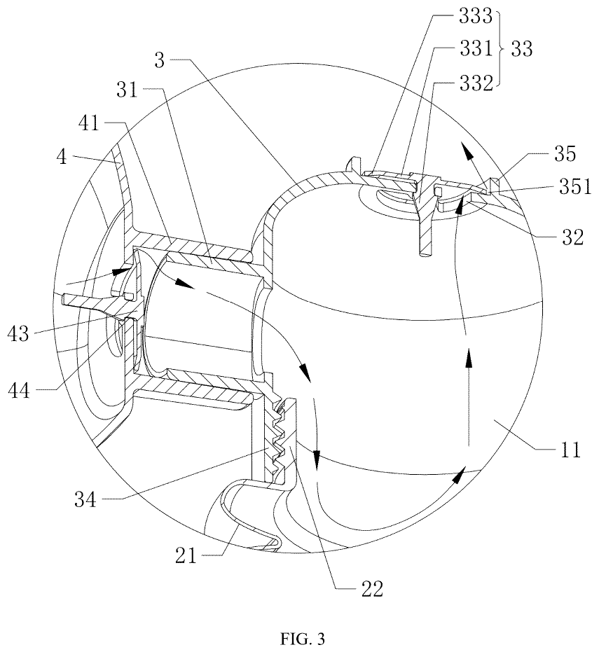

As shown in FIGS. 2-3, the cylinder body 2 includes a hard threaded connecting part 22 integrally connected with the cylinder shell 21, the cover body 3 is integrally provided with a threaded matching part 34 corresponding to the hard threaded connecting part 22, and the hard threaded connecting part 22 is in threaded connection with the threaded matching part 34, so that the cylinder body 2 and the cover body 3 can be quickly disassemble or assembled; both the one-way exhaust valve 33 and the one-way suction valve 43 include a diaphragm 331 and a mandrel 332 integrally connected to a middle of the diaphragm 331, and the mask 4 and the cover body 3 are provided with installation holes 44 corresponding to the mandrels 332, so that the mandrels 332 are movably arranged in the corresponding installation holes 44; a limiting part is arranged at an end part of each of the mandrels 332, the mandrels 332 can be prevented from falling out of the installation holes 44 by using the stopping function of the limiting part; the efficient sealing of the one-way exhaust valve or the one-way suction valve can be realized by using the elastic deformation capability and better sealing adhesion of the diaphragm 331; an annular flange 333 is obliquely arranged on a periphery of the diaphragm 331 in an extending manner toward the cover body 3 or the mask 4, and the addition of the annular flange 333 can prevent the situation that the diaphragm 331 is difficult to separate because the diaphragm 331 is attached to the surface of the mask 4 or the cover body 3 in a large area; the cover body 3 is provided with an annular protective sleeve 35 around the exhaust hole 32, the one-way exhaust valve 33 is arranged in the annular protective sleeve 35 and is in shielding fit with the corresponding exhaust hole 32, and an airflow gap 351 is reserved between a side wall of the one-way exhaust valve 33 and the annular protective sleeve 35, the annular protective sleeve 35 can protect the one-way exhaust valve 33, and meanwhile, the reservation of the airflow gap 351 can also ensure that the airflow in the cylinder body 2 can be smoothly exhausted from the exhaust hole 32; and in present embodiment, the membrane 331 of the one-way exhaust valve 33 is attached to a top of the cover body 3, and the diaphragm 331 of the one-way exhaust valve 43 is attached to an outer wall of the mask 4.

The basic working principle of the present invention is as follows: the exhaust hole 32 and the one-way exhaust valve 33 are arranged on the end face of one side of the cover body 3 far away from the cylinder body 2, and the suction pipe 31 is integrally arranged on the side wall of the cover body 3, so that this layout is favorable for a user to observe the use condition of the one-way exhaust valve 33 in real time when the anti-asphyxiation device is used for self-rescue, and interference impurities near the exhaust hole 32 can be conveniently cleaned by the user in time. Meanwhile, when the one-way exhaust valve 33 is damaged, the exposed one-way exhaust valve 33 is easier to replace. In addition, when the device is stored, the cylinder shell 21 can be compressed to be folded for storage, so that the space occupied when the device is stored is greatly reduced. The device has the effects that a user can conveniently observe the real-time working condition of the one-way exhaust valve, the one-way exhaust valve can be conveniently and quickly replaced or cleaned, and the space occupied when the device is stored is reduced.

### Embodiment 2

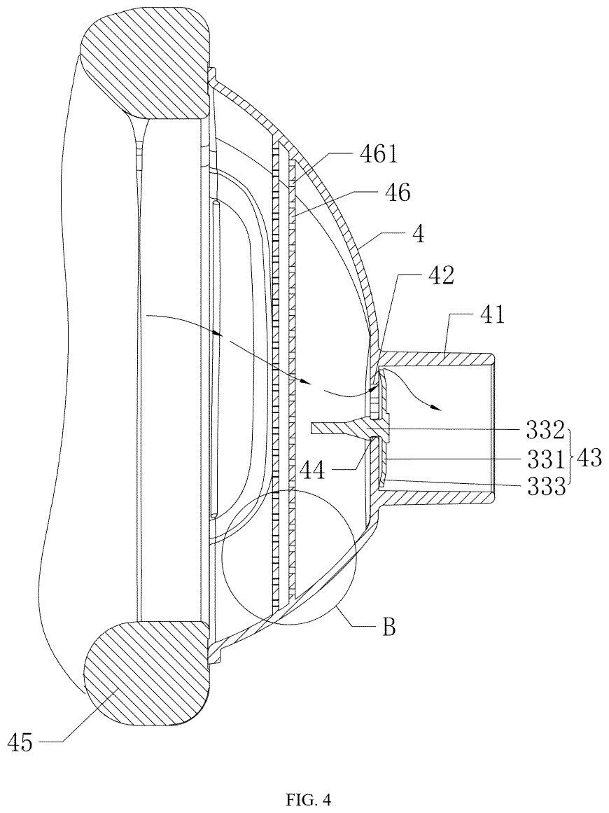

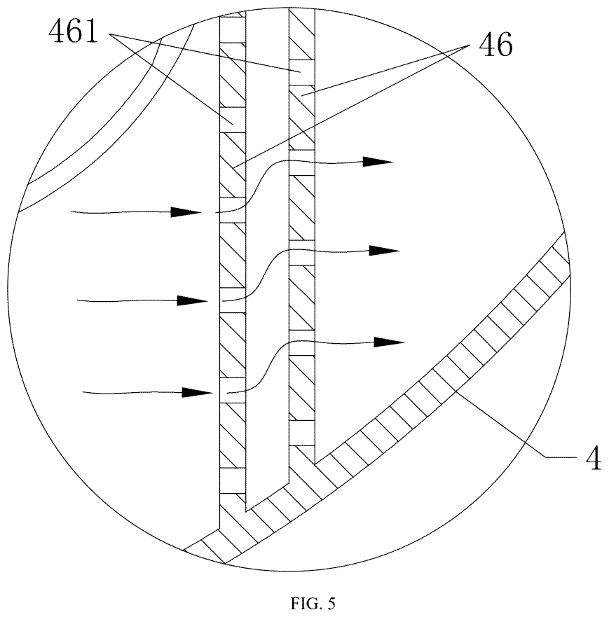

As shown in FIGS. 4-5, in the self-rescue anti-asphyxiation device, the difference between the present embodiment and the embodiment 1 is that the mask 4 is internally provided with a plurality of filter plates 46, each of the filter plates 46 is provided with a plurality of air passage holes 461, and the addition of the filter plates 46 can prevent insects or dust from entering the cylinder body 2, and meanwhile, if a user sneezes when wearing the mask 4, foreign matters can be effectively blocked on one side of the mask 4 far away from the cylinder body 2. In the present embodiment, two filter plates 46 are provided, the two filter plates 46 are arranged in the mask 4 at an interval, and the air passage holes 461 on the two filter plates 46 are arranged in a staggered manner, so that the blocking property of the filter plates 46 to the foreign matters can be further improved.

The present embodiment is consistent with other structures of the embodiment 1, which is not described here again.

The above is merely a preferred embodiment of the present invention, so all equivalent changes or modifications made according to the structure, features and principles described in the scope of the patent application of the present invention are included in the scope of the patent application of the present invention.

What is claimed is:

1. A self-rescue anti-asphyxiation device, comprising a suction main body (1) and a mask (4), wherein the suction main body (1) comprises a cylinder body (2) and a cover body (3) which are hermetically connected, the cylinder body (2) comprises a cylinder shell (21) having an elastic folding capability, a suction chamber (11) is formed between the cylinder body (2) and the cover body (3), the cover body (3) is provided with a suction pipe (31) and an exhaust hole (32) which are communicated with the suction chamber (11), the cover body (3) is provided with a one-way exhaust valve (33) corresponding to the exhaust hole (32), the exhaust hole (32) and the one-way exhaust valve (33) are arranged on an end face of one side of the cover body (3) far away from the cylinder body (2), and the suction pipe (31) is integrally arranged on a side wall of the cover body (3); and
the mask (4) is provided with a connecting sleeve (41), the connecting sleeve (41) is fixedly connected with the suction pipe (31) in an inserting manner, the mask (4) is provided with a suction hole (42) communicated with an inner cavity of the connecting sleeve (41) in a penetrating manner, and the mask (4) is provided with a one-way suction valve (43) corresponding to the suction hole (42).

2. The self-rescue anti-asphyxiation device according to claim 1, wherein the cylinder body (2) comprises a hard threaded connecting part (22) integrally connected with the cylinder shell (21), and the cover body (3) is integrally provided with a threaded matching part (34) corresponding to the hard threaded connecting part (22), and the hard threaded connecting part (22) is in threaded connection with the threaded matching part (34).

3. The self-rescue anti-asphyxiation device according to claim 1, wherein both the one-way exhaust valve (33) and the one-way suction valve (43) comprise a diaphragm (331) and a mandrel (332) integrally connected to a middle of the diaphragm (331), the mask (4) and the cover body (3) are provided with installation holes (44) corresponding to the mandrels (332), and the mandrels (332) are movably arranged in the corresponding installation holes (44).

4. The self-rescue anti-asphyxiation device according to claim 3, wherein an annular flange (333) is obliquely arranged on a periphery of the diaphragm (331) in an extending manner toward the cover body (3) or the mask (4).

5. The self-rescue anti-asphyxiation device according to claim 3, wherein the cover body (3) is provided with an annular protective sleeve (35) around the exhaust hole (32), the one-way exhaust valve (33) is arranged in the annular protective sleeve (35) and is in shielding fit with the corresponding exhaust hole (32), and an airflow gap (351) is reserved between a side wall of the one-way exhaust valve (33) and the annular protective sleeve (35).

6. The self-rescue anti-asphyxiation device according to claim 1, wherein the cylinder shell (21) is designed as a rubber bellows.

7. The self-rescue anti-asphyxiation device according to claim 1, wherein a handle part (23) is integrally connected to one end of the cylinder shell (21) far away from the cover body (3).

8. The self-rescue anti-asphyxiation device according to claim 1, wherein a face fitting ring (45) made of a flexible material is connected to an open end of the mask (4).

9. The self-rescue anti-asphyxiation device according to claim 1, wherein the mask (4) is internally provided with a plurality of filter plates (46), and each of the filter plates (46) is provided with a plurality of air passage holes (461).

10. The self-rescue anti-asphyxiation device according to claim 9, wherein two filter plates (46) are provided, the two filter plates (46) are arranged in the mask (4) at an interval, and the air passage holes (461) on the two filter plates (46) are arranged in a staggered manner.
